(19) United States
(12) Patent Application Publication
(10) Pub. No.: US 2020/0061149 A1
(43) Pub. Date: Feb. 27, 2020

(54) COMPOSITIONS AND METHODS FOR TREATING HEART FAILURE

(71) Applicants: The George Washington University, Washington, DC (US); The U.S. Government as represented by The Department of Veterans Affairs, Washington, DC (US)

(72) Inventors: Matthew Kay, Kensington, MD (US); Kara Garrott, Washington, DC (US); Jhansi Dyavanapalli, Gaithersburg, MD (US); David Mendelowitz, Vienna, VA (US); Trachiotis Gregory, Arlington, VA (US)

(21) Appl. No.: 16/563,635

(22) Filed: Sep. 6, 2019

Related U.S. Application Data
(63) Continuation of application No. PCT/US18/26583, filed on Apr. 6, 2018.
(60) Provisional application No. 62/482,983, filed on Apr. 7, 2017.

(51) Int. Cl. A61K 38/095; A61K 9/00; A61P 9/10

(52) U.S. Cl. CPC A61K 38/095; A61P 9/10; A61K 9/0043

(57) ABSTRACT

Methods of treating a subject having heart failure including heart failure with reduced ejection fraction, heart failure with preserved ejection fraction, and left ventricular hypertrophy-induced heart failure. The methods include activating hypothalamic oxytocin neurons in the brain of the subject and/or administering intranasally to the subject a therapeutically effective amount of oxytocin. Intranasal formulations for the treatment of a subject diagnosed with heart failure are also provided.

### CROSS REFERENCE TO RELATED APPLICATIONS

This application is a continuation of PCT Application No. PCT/US18/26583, entitled “Compositions and Methods for Treating Heart Failure,” filed Apr. 6, 2018, which claims the benefit of priority from U.S. Provisional Application No. 62/482,983, entitled “Chronic Activation of Hypothalamic Oxytocin Neurons Improves Cardiac Function During Left Ventricular Hypertrophy-Induced Heart Failure,” filed Apr. 7, 2017, both of which are incorporated by reference in their entireties, for all purposes, herein.

### STATEMENT OF GOVERNMENT SUPPORT

The present disclosure was made with government support under grant nos. R01-HL095828 and R01-HL133862 awarded by the National Institutes of Health. The government has certain rights in the present disclosure.

### FIELD

The present disclosure relates to the treatment of heart failure. In particular, the present disclosure relates to compositions and methods for treating patients diagnosed with heart failure, including, for example, Left Ventricular Hypertrophy (LVH), Heart Failure with Reduced Ejection Fraction (HFrEF), and Heart Failure with Preserved Ejection Fraction (HFpEF), by chronic activation of hypothalamic oxytocin neurons and/or intranasal administration of oxytocin.

### BACKGROUND

Heart failure (HF) affects 5.7 million adults in the United States and prevalence is projected to increase 46% in the next 15 years. Approximately 50% of patients diagnosed with HF die within 5 years, necessitating the development of new treatments. A hallmark of HF is elevated cardiac sympathetic activity and parasympathetic withdrawal, an imbalance that contributes to ventricular dysfunction, structural remodeling, and electrical instability. In the initial stages of HF, parasympathetic tone decreases as early as 3 days after the development of cardiac dysfunction, typically preceding increases in sympathetic activity. Elevated sympathetic activity is often managed with β-blockers, which alleviate HF symptoms; however, β-blockade does not address the functionally important reduction of cardiac parasympathetic tone that occurs with HF.

Previous studies have demonstrated the benefit of vagal nerve stimulation (VNS) to elevate parasympathetic tone during HF. Stimulation of the right vagus nerve during HF in rats improved LV function, prevented contraction deficits, reduced ventricular weight, and increased survival. In humans, clinical trials with left or right VNS demonstrated improved heart rate variability and six-minute walk distance in HF patients, but also indicated adverse effects that include dysphonia, cough, and throat pain that was likely due to the non-specificity of electrical VNS. A disadvantage of using electrical current to activate the vagus nerve is that although efferent cardiac fibers are activated, efferent fibers that innervate non-cardiac visceral organs, as well as sensory afferent fibers, are likely also activated. The efficacy of VNS is also dependent upon proper tuning of the stimulating current amplitude and frequency and maximum efficacy might require implantation of cuff electrodes around both the right and left vagus nerve. Approaches that selectively activate only cardiac parasympathetic neurons without the associated confounding variables and side effects that occur with VNS are desirable.

### BRIEF DESCRIPTION OF THE DRAWINGS

In order to describe the manner in which the advantages and features of the disclosure can be obtained, reference is made to embodiments thereof which are illustrated in the appended drawings. Understanding that these drawings depict only exemplary embodiments of the disclosure and are not therefore to be considered to be limiting of its scope, the principles herein are described and explained with additional specificity and detail through the use of the accompanying drawings in which:

FIG. 1 depicts images of representative H&E stained longitudinal slices of Control, TAC, and TAC+OXT hearts providing histological analysis of myocyte hypertrophy and fibrosis (n=6/group, 2 slides each), according to an exemplary embodiment of the present disclosure;

FIG. 2 depicts images (20× magnification) of H&E and Trichrome-stained transverse sections (LV free wall) from Control, TAC, and TAC+OXT hearts with higher collagen content (blue) evident in the TAC heart, according to an exemplary embodiment of the present disclosure;

FIG. 3 is a data plot depicting myocyte hypertrophy (n=6/group, 2 slides each) demonstrating that LV myocyte CS area was significantly greater in TAC compared to Control, TAC+OXT, and OXT NORM hearts (p<0.05), according to an exemplary embodiment of the present disclosure;

FIG. 4 is a data plot depicting myocyte hypertrophy (n=6/group, 2 slides each) demonstrating that LV collagen content was significantly higher in TAC compared to Control, TAC+OXT, and OXT NORM hearts (p<0.05), according to an exemplary embodiment of the present disclosure;

FIG. 5 depicts images of representative hearts for Control, TAC, and TAC+OXT hearts and transverse sections for Control, TAC, TAC+OXT, and OXT NORM hearts, demonstrating that PVN OXT neuron activation reduces morphological changes during TAC-induced pressure overload, according to an exemplary embodiment of the present disclosure;

FIG. 6 is a data plot depicting body weight for Control, TAC, TAC+OXT, and OXT NORM hearts and demonstrating that body weight 8 weeks after TAC was substantially the same between the groups, according to an exemplary embodiment of the present disclosure; and

FIG. 7 is a data plot depicting heart weight for Control, TAC, TAC+OXT, and OXT NORM hearts, and demonstrating that heart weight was not substantially different between Control (n=7), TAC (n=8), and TAC+OXT (n=9) hearts (ns), according to an exemplary embodiment of the present disclosure;

FIG. 8 is a data plot depicting LV free wall thickness for Control, TAC, TAC+OXT, and OXT NORM hearts, and demonstrating that the LV free wall was thicker in TAC (n=9) and TAC+OXT (n=9) hearts than in Control (n=7) and OXT NORM (n=6) hearts, according to an exemplary embodiment of the present disclosure;

FIG. 9 is a data plot depicting septum wall thickness for Control, TAC, TAC+OXT, and OXT NORM hearts, and demonstrating that the septal wall was thicker in TAC hearts (n=9) than Control (n=7), TAC+OXT (n=9), and OXT NORM (n=6) (p<0.05), according to an exemplary embodiment of the present disclosure;

FIG. 10 is a data plot depicting blood plasma OXT for Control, TAC, TAC+OXT, and OXT NORM hearts, and demonstrating that plasma OXT was not different between any group (ns, Control, TAC, OXT NORM: n=5; TAC+OXT: n=6) and indicating that the observed beneficial effects are due to specific activation of PVN OXT neurons and not global increases in blood OXT, according to an exemplary embodiment of the present disclosure;

FIG. 11 depicts IL-1β band intensities for Western blot assays for Control, TAC, TAC+OXT, and OXT NORM hearts revealing significant elevation of IL-1β in TAC hearts compared to Control, TAC+OXT, and OXT NORM hearts (p=0.0001, n=5/group), in which representative blots for IL-1β and GAPDH for each group are shown on the right, according to an exemplary embodiment of the present disclosure;

FIG. 12 depicts collagen III band intensities for Western blot assays for Control, TAC, TAC+OXT, and OXT NORM hearts, in which representative blots for collagen III and GAPDH for each group are shown on the right, and showing that collagen III protein expression was not different between the groups, however, collagen III expression in TAC and TAC+OXT hearts trended higher (ns, n=6/group), according to an exemplary embodiment of the present disclosure;

FIG. 13A is a data plot depicting representative in vivo arterial pressure in Control and DREADDs activated animals, according to an exemplary embodiment of the present disclosure;

FIG. 13B is a data plot depicting representative ECG measured from implanted telemetry device in Control and DREADDs activated animals, according to an exemplary embodiment of the present disclosure;

FIG. 14A is a data plot depicting decrease in blood pressure (BP) for CNO, CNO+atropine, and CNO+atropine+atenolol and demonstrating that acute activation of PVN OXT neurons by CNO significantly reduced BP (n=4, p<0.001), according to an exemplary embodiment of the present disclosure;

FIG. 14B is a data plot depicting decrease in heart rate (HR) for CNO, CNO+atropine, and CNO+atropine+atenolol and demonstrating that acute activation of PVN OXT neurons by CNO significantly reduced HR (n=4, p<0.001), according to an exemplary embodiment of the present disclosure;

FIG. 15A is a data plot depicting sinus rhythm in beats per minute (bpm) for Control, TAC, TAC+OXT, and OXT NORM hearts, and demonstrating that sinus rate in ex vivo hearts was not different among groups (p>0.05), according to an exemplary embodiment of the present disclosure;

FIG. 15B is a data plot depicting coronary flow rate (CFR) in mL/min for Control, TAC, TAC+OXT, and OXT NORM hearts, and demonstrating that CFR trended lower in TAC hearts (n=5) in comparison with Control (n=5), TAC+OXT (n=6), and OXT NORM (n=6) hearts (ns), according to an exemplary embodiment of the present disclosure;

FIG. 15C is a data plot depicting LV developed pressure (LVDP) in mmHg for Control, TAC, TAC+OXT, and OXT NORM hearts, and LVDP was significantly higher in Control (n=5), TAC+OXT (n=7), and OXT NORM (n=6) than TAC (n=6) hearts (p<0.05), according to an exemplary embodiment of the present disclosure;

FIG. 15D is a data plot depicting rate pressure product (RPP) in mmHg·bpm for Control, TAC, TAC+OXT, and OXT NORM hearts, and demonstrating that RPP was significantly depressed in TAC hearts compared to all other groups (p<0.05), according to an exemplary embodiment of the present disclosure;

FIG. 16 depicts representative LVDP signals for Control, TAC, TAC+OXT, and OXT NORM hearts, and demonstrating the reduced function of untreated TAC hearts, according to an exemplary embodiment of the present disclosure;

FIG. 17A is a data plot depicting LV contractility for Control, TAC, TAC+OXT, and OXT NORM hearts, and demonstrating that LV contractility was significantly greater in Control (n=5), TAC+OXT (n=7), and OXT NORM (n=6) compared to untreated TAC hearts (n=7) (p<0.05), according to an exemplary embodiment of the present disclosure;

FIG. 17B is a data plot depicting LV relaxation for Control, TAC, TAC+OXT, and OXT NORM hearts, and demonstrating that LV relaxation was significantly greater in Control (n=5), TAC+OXT (n=7), and OXT NORM (n=6) compared to untreated TAC hearts (n=7) (p<0.05), according to an exemplary embodiment of the present disclosure;

FIG. 18A depicts a data plot of heart rate (HR) in beats per minute (bpm) for Control, TAC, and TAC+OXT hearts, and demonstrating that HR was significantly different between all groups and between isoproterenol doses (p<0.05, GLM and Tukey pairwise analysis), according to an exemplary embodiment of the present disclosure;

FIG. 18B depicts a data plot of coronary flow rate (CFR) in mL/min for Control, TAC, and TAC+OXT hearts, and demonstrating that CFR was not different between control (n=5) and TAC+OXT (n=5) hearts, but was significantly lower in untreated TAC hearts (n=5) (GLM and Tukey pairwise comparisons, p<0.05), according to an exemplary embodiment of the present disclosure;

FIG. 18C depicts a data plot of rate pressure product (RPP) in mmHg·bpm (thousandths) for Control, TAC, and TAC+OXT hearts, and demonstrating that on average, RPP increased in all hearts with increased isoproterenol concentration and was significantly different between all groups (p<0.05, GLM and Tukey pairwise analysis), according to an exemplary embodiment of the present disclosure;

FIG. 19A depicts a data plot of contractility (mmHg·bpm) versus isoproterenol dose (nM) for Control, TAC, and TAC+OXT hearts, according to an exemplary embodiment of the present disclosure; and

FIG. 19B depicts a data plot of relaxation (mmHg·bpm) versus isoproterenol dose (nM) for Control, TAC, and TAC+OXT hearts, according to an exemplary embodiment of the present disclosure.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure.

It should be understood at the outset that although illustrative implementations of one or more embodiments are illustrated below, the disclosed compositions and methods may be implemented using any number of techniques. The disclosure should in no way be limited to the illustrative implementations, drawings, and techniques illustrated herein, but may be modified within the scope of the appended claims along with their full scope of equivalents.

In the following discussion and in the claims, the terms “including” and “comprising” are used in an open-ended fashion, and thus should be interpreted to mean “including, but not limited to . . . ”. As used herein, the term “heart failure,” in all its forms refers to a condition in a subject classified according to any one of the New York Heart Association (NYHA) functional classes, including Class I, Class II, Class III, or Class IV. As used herein, the term “heart failure with preserved ejection fraction,” in all its forms, including “HFpEF” or “HFPEF,” refers to a condition in a subject having heart failure in which the subject has a left ventricular ejection fraction that is greater than 50%. As used herein, the term “heart failure with preserved ejection fraction,” may be used, in at least some instances, interchangeably with the terms “diastolic heart failure” and “diastolic dysfunction.”

As used herein, the term “hypertrophy of the heart,” in all its forms, refers to a condition in which the heart muscle, or any portion thereof, is determined to be abnormally thick as measured by electrocardiogram and/or echocardiology. As used herein, the term “left ventricular hypertrophy (LVH),” in all its forms, refers to a condition in which the walls of the left ventricle of the heart are greater than 1.5 centimeters as measured by echocardiogram. As used herein, the term “IU,” in all its forms, used with respect to the dosage of oxytocin, is equivalent to 2 μg of pure peptide. As used herein, the term “improving cardiac function” refers to an improvement in the ability of the heart to contract and/or relax, increased coronary blood flow, providing favorable remodeling in a subject with heart failure, decreasing fibrosis, decreasing the hypertrophy of cardiac myocytes, improving calcium handling in myocytes in a heart failure subject, or any combination thereof.

The various characteristics described in more detail below, will be readily apparent to those skilled in the art with the aid of this disclosure upon reading the following detailed description, and by referring to the accompanying drawings.

The present disclosure provides methods and formulations for treating subjects having heart failure and related conditions by selectively activating cardiac vagal neurons (CVNs) in the dorsal motor nucleus of the vagus and/or Nucleus Ambiguus to achieve the benefits of left and right VNS without the side effects of electrical stimulation. The presently disclosed techniques and compositions may form the basis of a new clinical therapy for the treatment of heart failure and related conditions. CVNs within the brainstem regulate parasympathetic activity to the heart to maintain normal heart rate (HR) and coronary flow. CVNs receive powerful excitation from a population of oxytocin (OXT) neurons within the paraventricular nucleus (PVN) of the hypothalamus that co-release OXT and glutamate to excite CVNs. This higher brain center is responsible for regulating both autonomic function in normal situations and cardiac responses in high-stress conditions. We have discovered that in rats with LV hypertrophy that progresses to HF, CVNs have diminished excitation due to both an increase in spontaneous inhibitory GABAergic neurotransmission frequency and a decrease in amplitude and frequency of excitatory glutamatergic neurotransmission to CVNs. This finding indicates that augmentation of the excitatory PVN OXT/glutamate pathway to CVNs may be a promising approach to maintain cardiac parasympathetic activity during HF.

OXT is important for maintaining cardiovascular homeostasis and parasympathetic cardiac activity, particularly during anxiety and stress. For example, OXT administration may prevent increased HR and diminished HR variability that occurs with social isolation. Additionally, rats subjected to daily restraint stress have increased cardiac infarct size and increased incidence of severe arrhythmias during myocardial ischemia-reperfusion, while intra-cerebroventricular administration of OXT, that did not increase plasma OXT levels, reduced the cardiac injury that occurred following episodes of ischemia reperfusion. We have discovered that chronic activation of PVN OXT neurons in rats restores the release of OXT from PVN fibers, decreases heart rate and blood pressure, and more importantly, prevents the hypertension that occurs during chronic intermittent hypoxia/hypercapnia. In particular, the present disclosure demonstrates that chronic activation of hypothalamic PVN OXT neurons in a rat model of HF (TAC) prevents loss of cardiac contractile function and reduces cardiac inflammation and fibrosis compared to age-matched untreated HF rats (Control). Designer Receptors Specifically Activated by Designer Drugs (DREADDs) were selectively expressed in hypothalamic PVN OXT neurons of all animals. DREADDs were activated with daily injections of clozapine N-oxide (CNO) in the treatment HF group (TAC+OXT) and in a control group (OXT NORM). LV function, LV fibrosis, and the expression level of the inflammatory cytokine interleukin-β (IL-β) were assessed via excised perfused heart experiments, histology, and western blot assays. Our results indicate that chronic activation of hypothalamic OXT neurons could be an effective approach to slow the development of cardiac damage and dysfunction that occurs during pressure overload HF. As a result, chronic activation of PVN OXT neurons may increase cardiac parasympathetic activity and blunt the progression of cardiac dysfunction during HF.

According to an aspect of the present disclosure, a method of treating a subject having heart failure is provided. The subject may be a mammalian subject or a human subject. The method may include administering intranasally to the subject a therapeutically effective amount of oxytocin. The therapeutically effective amount of oxytocin may be from about 20 IU to about 100 IU, or from about 20 IU to about 60 IU, or from about 10 IU to about 100 IU, or from about 20 IU to about 40 IU, or from about 30 IU to about 50 IU. In at least some instances, the pharmaceutically effective amount is from about 20 IU to about 40 IU b.i.d.

The therapeutically effective amount of oxytocin may be administered once per day or twice per day. In at least some instances, the pharmaceutically effective amount of oxytocin may be administered at least once a day for at least 5 days, or at least 10 days, or at least one month, or at least 6 months. In some cases, the pharmaceutically effective amount of oxytocin may be administered at least once a day for consecutive days or may be administered at least once a day chronically or for the remainder of the subject's life. In at least some instances, the pharmaceutically effective amount of oxytocin may be administered twice a day for at least 5 days, or at least 10 days, or at least one month, or at least 6 months. In some cases, the pharmaceutically effective amount of oxytocin may be administered twice a day for consecutive days or may be administered twice a day chronically or for the remainder of the subject's life.

In at least some instances, the method may further include administering to the subject a therapeutically effective amount of nitric oxide, atrial natriuretic peptide (ANP), and/or beta-blockers.

According to an aspect of the present disclosure, the presently disclosed methods and compositions may be used to treat a subject having heart failure as defined by NYHA Class I-IV classification. In at least some instances, the subject may have hypertrophy of the heart and/or a left ventricular fraction less than or equal to 40%. The subject may have both heart failure and hypertrophy of the heart. The subject may also have heart failure, including heart failure with preserved ejection fraction or heart failure with reduced ejection fraction, and also have one or more of hypertrophy of the heart, cardiac ischemia, left ventricular hypertrophy, and a left ventricular ejection fraction of less than or equal to 40%. In at least some instances, the subject does not have ischemic heart disease. In some instances, the subject may have heart failure with reduced ejection fraction. In other cases, the subject may have heart failure with preserved ejection fraction. In some instances, the subject may have left ventricular hypertrophy-induced heart failure. In at least some instances, the presently disclosed methods may be used to treat a subject having cardiac ischemia or a patient diagnosed with cardiac ischemia. For example, the subject may have cardiac ischemia due to coronary artery disease, one or more blood clots, or a coronary artery spasm.

According to one aspect of the present disclosure, a method of treating a subject diagnosed with heart failure that includes activating hypothalamic oxytocin neurons in the brain of the subject is provided. In at least some instances, the method includes chronic activation of PVN OXT neurons in the hypothalamus of the subject. According to the present disclosure, activation of hypothalamic oxytocin neurons in a subject may provide beneficial effects such as reduced heart rate, anti-inflammation, reduced fibrosis, and increased coronary flow.

In some instances, the hypothalamic oxytocin neurons in the brain of the subject may be activated by administering an effective amount of oxytocin to the subject. In some cases, the hypothalamic oxytocin neurons in the brain of the subject may be activated by intranasal administration of oxytocin to the subject. In other instances, the hypothalamic oxytocin neurons in the brain of the subject may be activated by viral mediated expression of exogenous receptors that can be activated by otherwise biologically inert agents—analogous to the currently used DREADDs approach in animal models.

According to an aspect of the present disclosure, an intranasal formulation for the treatment of a subject diagnosed with heart failure is provided. The formulation may include a therapeutically effective amount of oxytocin capable of being delivered intranasally to the subject. In at least some instances, the formulation may include a pharmaceutically acceptable carrier. The pharmaceutically acceptable carrier may be, for example, water, ethanol, propylene glycol, polyethylene glycol, vegetable oils, organic esters, glycerin, phenol, dimethyl sulfoxide, N-tridecyl-β-D-maltoside, and any combination thereof.

The formulation may be manufactured to supply oxytocin in an amount from about 10 IU to about 100 IU per each administration. In some instances, the formulation may be manufactured to supply oxytocin in an amount from about 20 IU to about 40 IU per each administration. In at least some cases, the formulation may be manufactured to supply oxytocin in an amount from about 20 IU to about 40 IU b.i.d. The formulation may be manufactured to supply oxytocin in an amount of about 40 IU per each administration. The formulation may be manufactured to be administered once per day or twice per day. In some instances, the formulation may further include a therapeutically effective amount of nitric oxide, atrial natriuretic peptide (ANP), and/or beta-blockers.

### EXAMPLES

### Example 1

### Chronic Activation of Hypothalamic PVN OXT Neurons in Rat Model of Heart Failure (TAC)

### Ethical Approval

All animal procedures were completed in agreement with the George Washington University institutional guidelines and in compliance with suggestions from the panel of Euthanasia of the American Veterinary Medical Association and the National Institutes of Health (NIH) Guide for the Care and Use of Laboratory Animals.

### Surgical Procedure for Trans-Ascending Aortic Constriction

Pressure overload induced LV hypertrophy was initiated in male Sprague-Dawley rats using a minimally invasive trans-ascending aortic constriction (TAC) procedure. Rats at one week of age were aestheticized by hypothermia and underwent TAC surgery. A 0.5 cm incision was made at the level of the chest, the chest was opened and the thymus was retracted to reveal the aorta. A 4-0 silk suture was passed around the ascending aorta, and with a 25-gauge needle temporarily placed adjacent to the aorta, the suture was tied around both the aorta and needle (TAC and TAC+OXT groups). The needle was then removed, leaving the constricting suture around the aorta. Buprenorphine was applied as an analgesic. Successful constriction was confirmed upon examination of the aorta after each animal was sacrificed.

### Selective Expression and Activation of DREADDs in PVN Oxytocin Neurons

Selective activation of PVN OXT neurons was achieved using DREADDs with a highly selective OXT promoter to drive expression of DREADDs in PVN OXT neurons. Injections of clozapine-N-oxide (CNO) increases the firing of PVN OXT neurons for at least one hour. Additionally, selective activation of DREADDs in PVN OXT neurons decreases both mean arterial pressure and HR in telemetry instrumented conscious unrestrained animals. To ensure robust and highly selective expression in PVN OXT neurons, two viral vectors were used in combination with the Cre-Lox recombination system. In this system one viral vector expresses Cre recombinase under an OXT promotor. The second vector expresses the excitatory hM3Dq DREADDs22. This is a Cre-dependent vector that has silencing double-floxed inverse open reading frames, which insures expression is only in OXT neurons that selectively express Cre. In each animal, 30-50 nl containing both viral vectors was selectively microinjected into the PVN over a 20 minutes period at 1 week of age.

### Activation of PVN Oxytocin Neurons In Vivo

PVN OXT neurons expressing DREADDs were exclusively activated by clozapine-N-oxide (CNO), a molecule that is otherwise biologically inert and does not cross the blood-brain-barrier. TAC+OXT and OXT NORM animals received intraperitoneal (IP) injections of CNO (1 mg/kg) daily, beginning at 5 weeks of age until the animal was sacrificed.

### In Vivo Assessments of Changes in Autonomic Tone Upon PVN OXT Neuron Activation

Sprague-Dawley rats at 4 weeks of age that had DREADDS expression in PVN oxytocin neurons were anesthetized (isoflurane) and implanted with a telemetry device (HDX-11, DSI) with the pressure catheter inserted into the descending abdominal aorta to measure blood pressure (BP). The ECG leads of the device were inserted subcutaneously to measure HR. Rats were allowed to recover from this surgery for a week, followed by IP saline injections for 3 days to acclimate the animals to IP injections. BP & HR signals were then recorded 15 minutes before and 1 hour after IP injections of either CNO (1 mg/kg), CNO (1 mg/kg)+atropine (1 mg/kg), or CNO (1 mg/kg)+atropine (1 mg/kg)+atenolol 10 mg/kg). One injection of one of the three solutions was administered each day on consecutive days.

### Ex Vivo Assessments of Cardiac Function

At nine weeks of age rats were anesthetized with an IP injection of 0.2 mL Telazol and subject to isoflurane inhalation. Following cessation of pain reflexes, hearts were rapidly excised (n=26) and Langendorff perfused via the aorta at constant pressure (65 mmHg) and temperature (37° C.) with a Krebs-Henseleit solution containing (in mM) 115 NaCl, 3.3 KCl, 2.0 CaCl₂, 1.4 MgSO₄, 25.0 NaHCO₃, 1.0 KH₂PO₄, 5.0 glucose, and 1.0 lactate. Perfusate was oxygenated with 95% O₂-5% CO₂. After a stabilization period of 10 minutes, a size 5 balloon (Harvard Apparatus) was inserted into the LV to measure isovolumic LV developed pressure (LVDP). Diastolic pressure was set to 10 mmHg and LVDP was computed as the difference between systolic and diastolic pressures. HR, LVDP, and coronary flow rate (CFR) were measured for at least 15 minutes during sinus rhythm. An isoproterenol (16504, Sigma Aldrich) dose response protocol was then administered using concentrations of 0.01, 0.1, 1, 10, 100, 1000 nM, and HR, LVDP, and CFR were measured at each concentration once function stabilized. After each study, rate pressure product (RPP), an indirect measure of LV work, was calculated as the product of HR and LVDP. Contractility and relaxation were measured as the maximum and minimum values of the first derivative of the LVP waveform, respectively, to assess inotropy and lusitropy.

### Anatomic Measurements and Histology

In a subset of animals, hearts were excised and cannulated via the aorta, flushed of blood, and weighed after clearing fluid from the chambers. The hearts were cut transversely, halfway between the apex and base, and the thickness of the LV free wall and septum were measured. The tissue was then preserved in 10% formalin for histology. Other hearts from each group were cannulated, flushed with high potassium solution, and then formalin-fixed via retrograde perfusion at constant pressure (60 mmHg). The hearts were cut longitudinally and preserved in 10% formalin for histology. All heart specimens were stained (H&E and Trichrome) to quantify cell size and collagen deposition. Average myocyte transverse cross-sectional (CS) area (μm²) and the amount of fibrosis (μm²) observed in a histological section were measured using ImageJ for at least three transverse slices from each heart.

### Measurement of Plasma OXT

At least 2 mL of blood was collected from each rat before heart excision. Blood was centrifuged (2000 g) at 4° C. for 15 minutes. Supernatant (plasma) was then decanted and stored at −80° C. Plasma OXT level was then measured for plasma samples from each group (n=5-6/group) by Cayman Chemicals using the Oxytocin ELISA Kit (Cayman Chemical Company).

### Western Blotting

Samples of LV myocardium (n=5-6/group) were flash frozen in liquid nitrogen and stored at −80° C. For western blot analysis of protein expression, samples were thawed and homogenized using the Qproteome Mammalian Protein Prep Kit (Qiagen) in tubes containing metallic beads. Samples were then centrifuged at 14,000g for 20 minutes and protein concentrations were determined by the Pierce BCA Protein Assay (Thermo Scientific). Laemmli buffer (Bio Rad) with 10% 2-mercaptoethanol (Sigma-Aldrich) was added to the samples and samples were heated to 98° C. Equal protein concentration was loaded into wells containing 4-20% Mini-PROTEAN® TGX™ Gels (Bio Rad). The samples were then run at 100V for 1-1.5 hours to separate proteins by electrophoresis.

After electrophoresis, the samples were transferred to PVDF membranes for 10 min at 25V using the Trans-Blot® Turbo™ Transfer System (Bio Rad). The membranes were blocked with 5% milk for 18 hours. The membrane was incubated for 1 hour at room temperature with one of three primary antibodies: collagen III (Abcam) 1:2000; interleukin-1β (IL-1β) (Cell Signaling Technology) 1:1000; or GAPDH (Sigma-Aldrich) 1:6000. The membranes were then washed and incubated for 1 hour with the HRP-conjugated secondary antibodies anti-rabbit 1:6000 and anti-mouse 1:5000 (Santa Cruz). The membranes were washed again and gels were imaged using the Azure cSeries c600 (VWR). Band intensities for collagen III and IL-1β were measured using ImageJ and normalized to the corresponding band intensity for GAPDH.

### Statistical Measurements

Measurements in each group were compared using analysis of variance with Tukey post-hoc comparisons (Minitab 17 Statistical Software) to identify significant differences between groups. Myocyte area measurements were analyzed using fully nested ANOVA to account for both number of cells and number of animals. The isoproterenol dose response data was analyzed using a general linear model (GLM) to evaluate the group effect and the isoproterenol dose effect. Tukey post-hoc tests identified pairwise changes between groups at each isoproterenol concentration. Differences were considered significant if p<0.05.

### Results

At 8 weeks post-TAC, ex vivo LV function and sensitivity to β-adrenergic stimulation was measured in isovolumic working heart experiments for animals in each group (control n=5; TAC n=7; TAC+OXT n=7; OXT NORM n=6). Additional hearts were prepared for histological analysis, as described above (Control n=5; TAC n=5; TAC+OXT n=5; OXT NORM n=3). Average myocyte transverse CS area, fibrosis, and anatomic differences between groups are listed in Table 1.

TABLE 1

| | Control | TAC | TAC + OXT | OXT NORM |
|---|---|---|---|---|
| Myocyte area (μm²) | 356 ± 98 | 713 ± 88* | 584 ± 51⁺ | 365 ± 90 |
| Fibrosis (μm²) | 980 ± 309 | 7749 ± 1273* | 2407 ± 723⁺ | 918 ± 112 |
| Body weight (g) | 350 ± 27 | 275 ± 17 | 298 ± 17 | 357 ± 24 |
| Heart weight (g) | 1.17 ± 0.10 | 1.62 ± 0.05* | 1.75 ± 0.21 | 2.19 ± 0.17* |
| LV wall thickness (mm) | 3.78 ± 0.25 | 5.87 ± 0.19*^{#} | 5.53 ± 0.34*^{#} | 3.69 ± 0.10 |
| Septum thickness (mm) | 3.75 ± 0.18 | 4.95 ± 0.23*^{+#} | 3.99 ± 0.14 | 3.18 ± 0.12 |

*different from Control
⁺different from TAC + OXT
^{#}different from OXT NORM

### Myocyte Hypertrophy and Myocardial Fibrosis

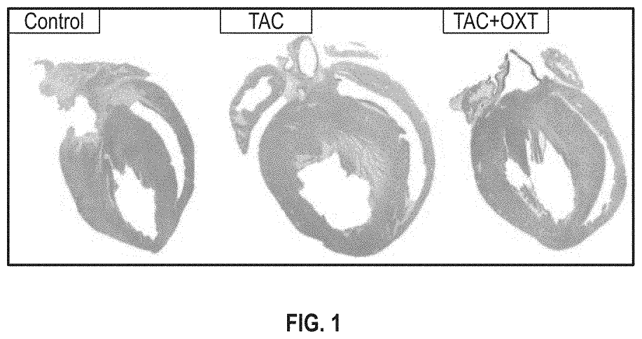

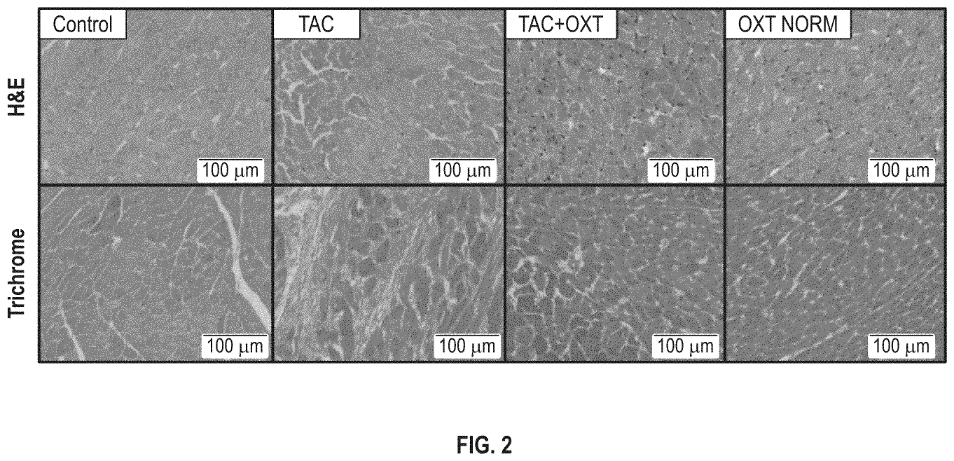

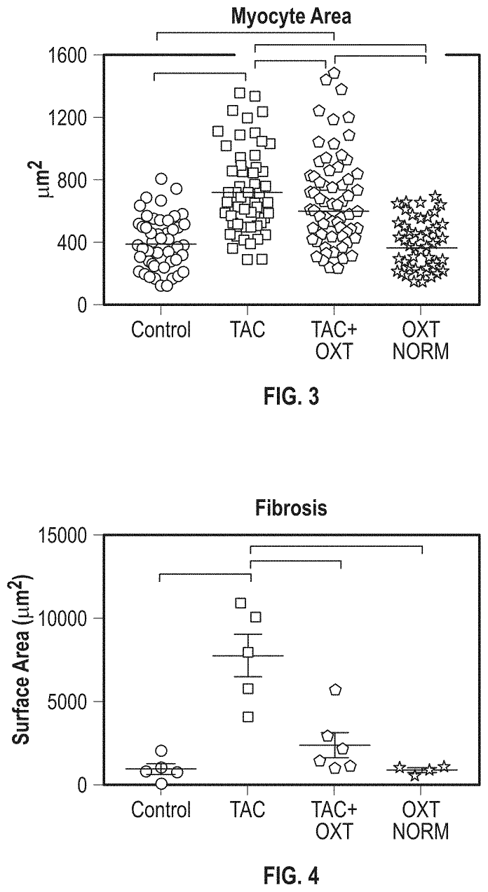

Images of typical H&E and Trichrome histological sections are shown in FIGS. 1-2. Higher collagen content (blue) is evident in the TAC heart. As depicted in FIG. 3, myocyte CS area was 389±20 μm² in Control hearts and was no different in OXT NORM hearts (368±20 μm²). Myocyte CS area was significantly higher in TAC hearts (720±35 μm²) compared to Control and TAC+OXT (601±23 μm²) hearts. As depicted in FIG. 4, hearts from TAC rats also developed significantly more fibrosis (7749±1273 μm²) than hearts from Control, TAC+OXT, and OXT NORM rats (980±309 μm², 2407±723 μm², and 918±112 μm² respectively).

### Anatomic Differences

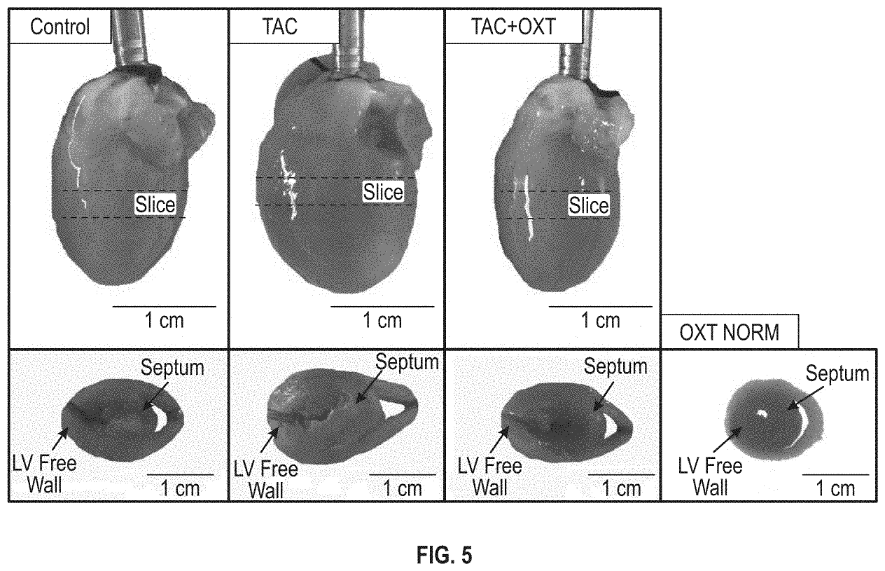

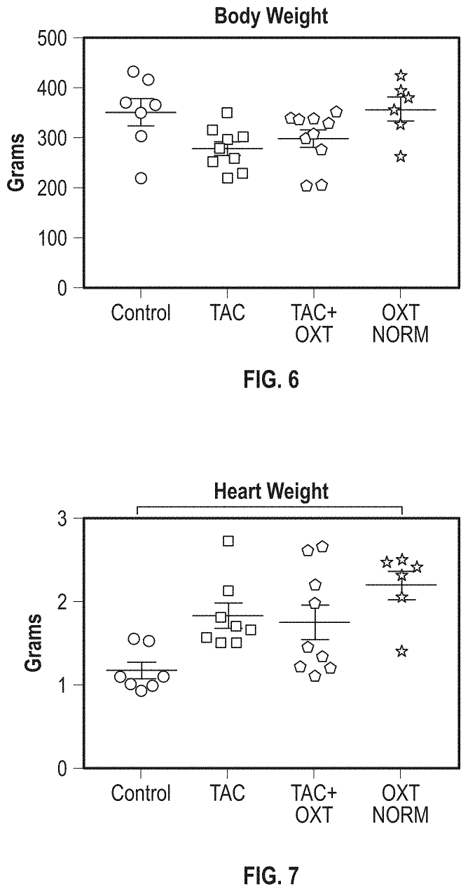

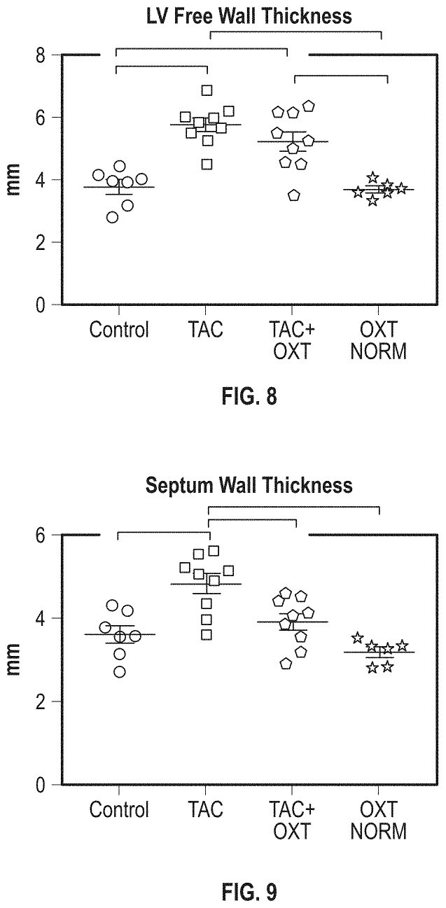

Typical transverse slices of hearts from animals of each group are shown in FIG. 5. There was no significant difference in body weight between groups at the time of sacrifice, as shown in FIG. 6. As depicted in FIG. 7, heart weight was not significantly different between Control (1.17±0.1 g), TAC (1.62±0.05 g) and TAC+OXT (1.74±0.2 g); however, OXT NORM (2.19±0.2 g) hearts weighed more than Control (p<0.05). LV free wall thickness, as depicted in FIG. 8, was greater than Control and OXT NORM (3.78±0.3 mm, 3.69±0.1 mm, respectively) in both TAC and TAC+OXT animals (5.87±0.2 mm, 5.22±0.3 mm, respectively). The wall thickness of the ventricular septum was significantly greater in TAC (4.95±0.2 mm) than all other groups (Control: 3.75±0.2 mm; TAC+OXT: 3.9±0.2 mm; OXT NORM: 3.18±0.1 mm), as shown in FIG. 9.

### Blood OXT Levels and Myocardial Levels of Collagen III and IL-1β

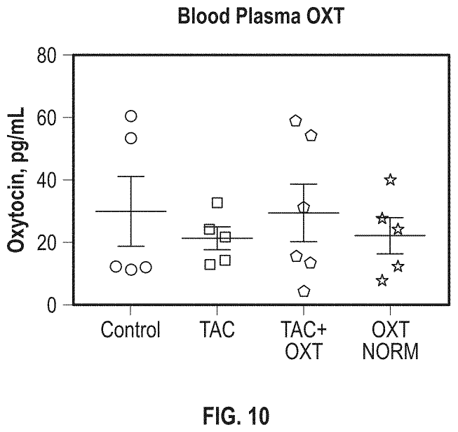

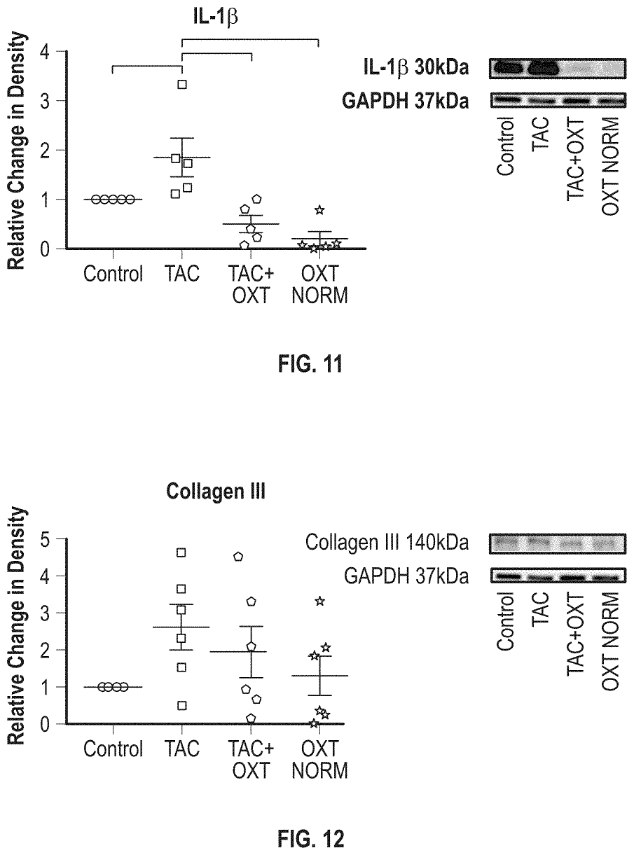

OXT neuron activation did not elevate plasma oxytocin levels. OXT neuron activation blunted cardiac levels of IL-1β, thereby reducing inflammation and blunting increased fibrosis. In particular, ELISA measurements of plasma OXT levels indicated no significant difference in plasma OXT between the four groups (p=0.827), as shown in FIG. 10. As depicted in FIG. 11, expression of proteins integral to inflammation (IL-1β) and fibrosis (collagen III) was measured by western blot assays and compared between groups to reveal a significant increase in IL-1β expression in TAC hearts. IL-1βexpression in TAC hearts was significantly elevated, 1.84±0.4 times higher than Control expression (p=0.0001). There was no difference in IL-1β expression between Control, TAC+OXT, and OXT NORM hearts (0.50±0.2, and 0.21±0.14 change from Control, respectively). As shown in FIG. 12, there was no difference in collagen III expression between the groups; however, TAC and TAC+OXT (2.6±0.6 and 1.95±0.7 times Control) collagen III expression trended greater than that of Control and OXT NORM (1.31±0.5 change from Control). Band intensities for collagen III and IL-1β were measured using ImageJ and normalized to the corresponding band intensity for GAPDH.

### In Vivo Assessments of Autonomic Tone

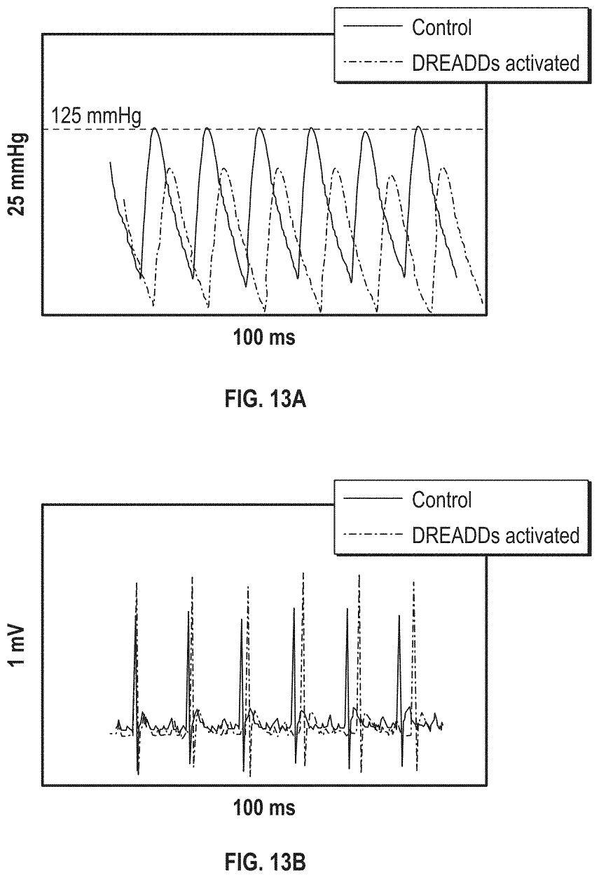

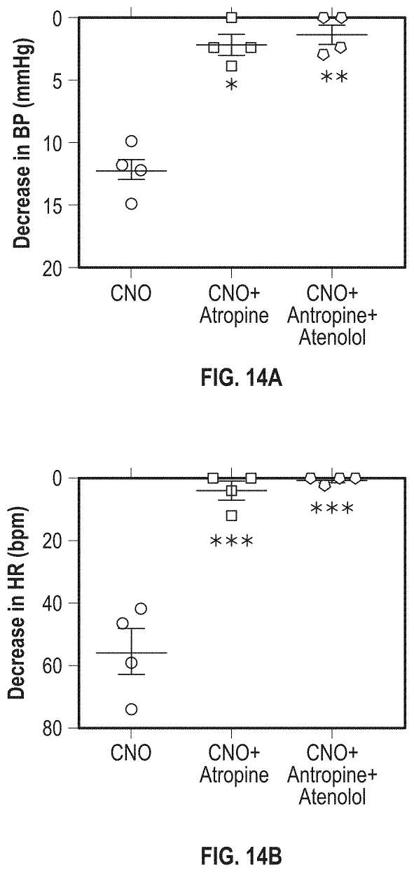

Acute activation of PVN OXT neurons by CNO in awake and conscience animals significantly reduced blood pressure an average of 13 mmHg (from 105.8±1.9 mmHg to 93.4±1.5 mmHg; n=4; p<0.001) and significantly reduced HR an average of 56 bpm (from 475.4±15.9 bpm to 419±12.4 bpm; n=4; p<0.001), as shown in FIGS. 13-14. The muscarinic receptor antagonist atropine prevented these responses. As depicted in FIGS. 14A and 14B, blocking β1 receptors with atenolol had no significant additional effects on the responses to PVN OXT neuron activation.

### Contractile Function of Excised Hearts

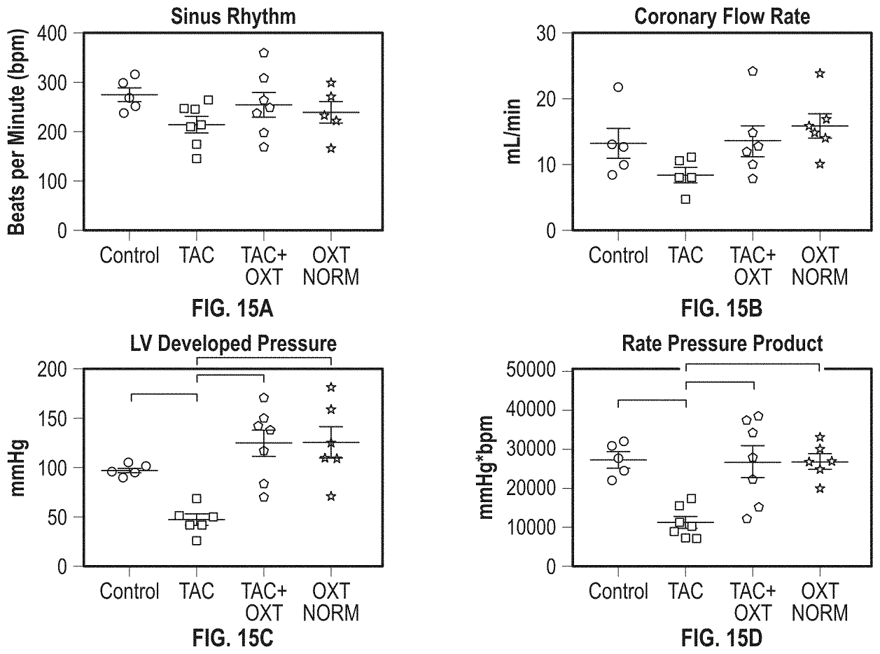

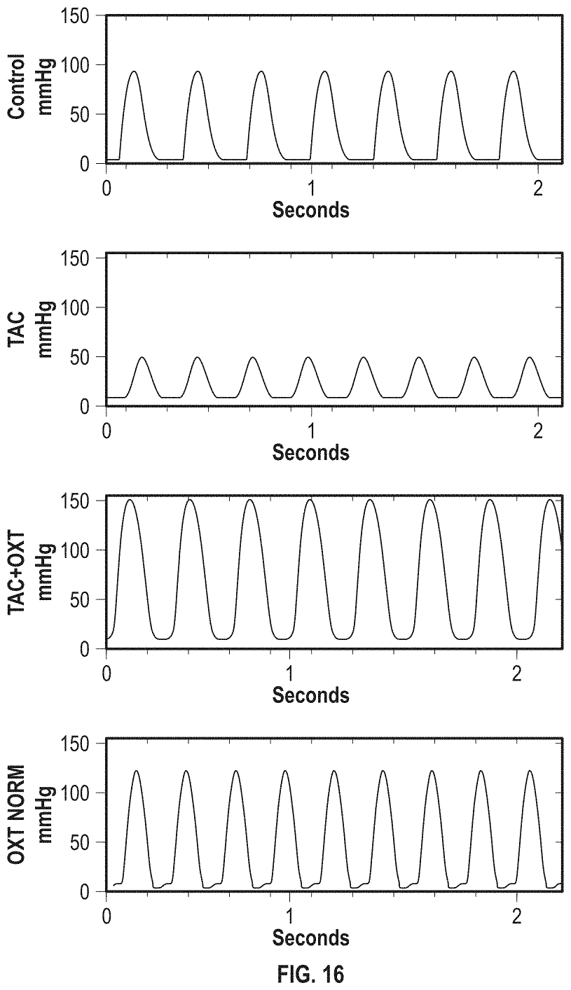

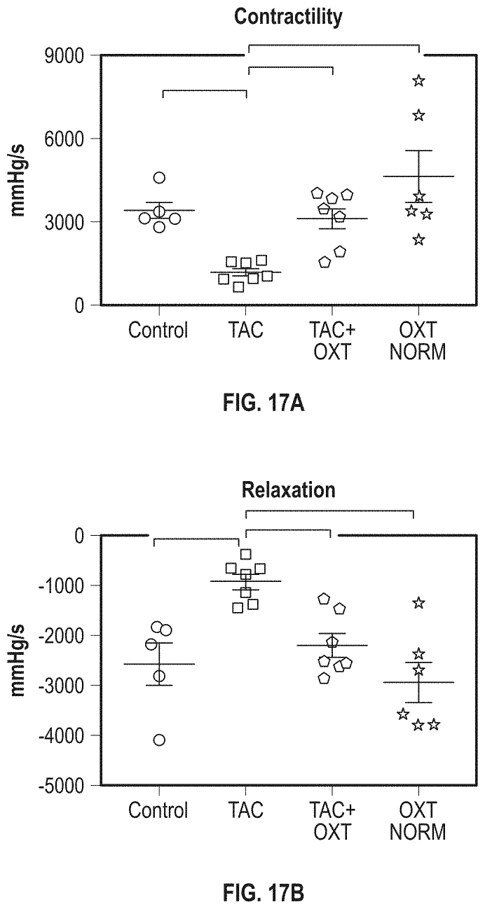

Hearts from TAC animals with PVN OXT treatment had significantly improved LV function above that of hearts from untreated TAC animals. The isovolumic contractile function of hearts excised from animals of each group was assessed during normal sinus rhythm, which was the same between groups, as shown in FIG. 15A. Of note, the LV function of TAC+OXT hearts closely matched that of Control hearts. CFR for TAC+OXT (13.8±2.9 mL/min) and OXT NORM hearts (16±1.9 mL/min) was similar to that of Control hearts (13.3±2.4 mL/min) while CFR trended lower for TAC hearts (8.4±1.1 mL/min), although the difference was not significant, as depicted in FIG. 15B. The LVDP of TAC hearts was significantly lower (52±7 mmHg) than that of Control, TAC+OXT, and OXT NORM hearts, as shown in FIG. 15C, which maintained average LVDPs of 98±3 mmHg, 126±14 mmHg, and 127±16 mmHg, respectively. RPP, an indirect measure of work, was also significantly lower for TAC hearts, as depicted in FIG. 15D, during sinus rhythm (11,081±1,612 mmHg*bpm) compared to Control (27,473±1,894 mmHg*bpm), TAC+OXT (26,874±4,036 mmHg*bpm), and OXT NORM (26,978±2,274 mmHg*bpm) hearts. FIG. 16 depicts representative LVDP signals for Control, TAC, TAC+OXT, and OXT NORM hearts, thereby demonstrating the reduced function of untreated TAC hearts. As depicted in FIGS. 17A and 17B, average contractility and relaxation for TAC hearts was also significantly less (1,165±121 mmHg/s and −917±144 mmHg/s) than Control (3,383±313 mmHg/s and −2,557±416 mmHg/s), TAC+OXT (3,124±383 mmHg/s and −2,203±231 mmHg/s), and OXT NORM (4,631±931 mmHg/s and −2,926±397 mmHg/s) hearts. These data clearly reveal improved LV function and potentially unimpaired coronary flow in animals treated with PVN OXT neuron activation.

### Heart Rate Sensitivity to β-Adrenergic Stimulation

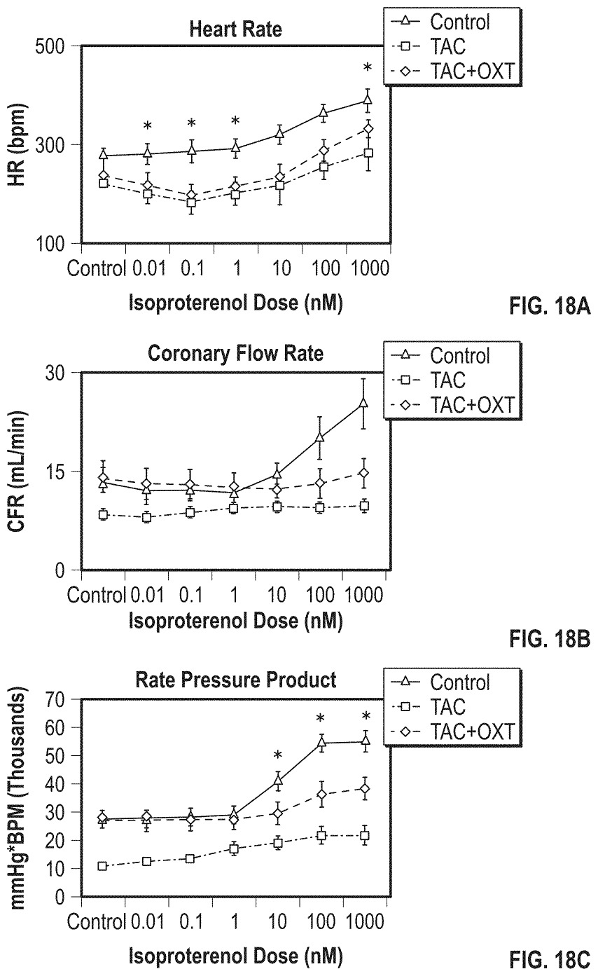

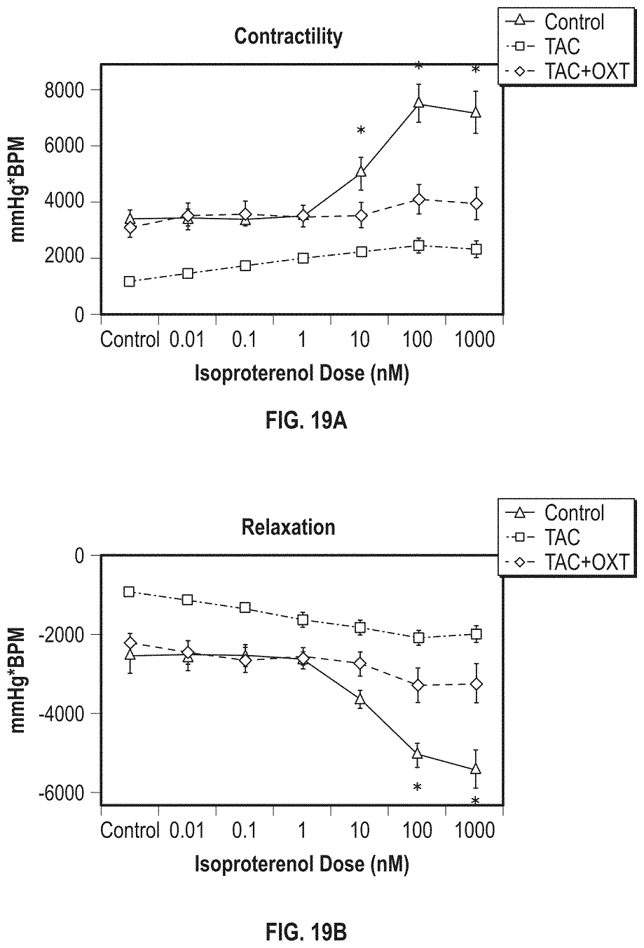

Results from the isoproterenol dose response studies are shown in FIGS. 18 and 19. Significant differences were not detected between Control and OXT NORM in any metric at the highest isoproterenol concentrations; therefore, OXT NORM data are not shown in FIGS. 18 and 19. As shown in FIG. 18, isoproterenol dose-response curves reveal that hearts from TAC+OXT animals had improved heart rate response to β-adrenergic sensitivity. Asterisks indicate significant differences between all groups at specific isoproterenol dose (GLM and Tukey pairwise analysis). Average HR for each group trended higher with increasing isoproterenol concentration and TAC+OXT hearts responded with the greatest increase in HR (p<0.05), as shown in FIG. 18A. Of note, baseline (no isoproterenol) HR for TAC+OXT hearts matched that of TAC hearts yet HR increased in TAC+OXT hearts with increasing isoproterenol concentration to match that of Control hearts at the highest isoproterenol concentration (1000 nM).

### LV Contractile Sensitivity to β-Adrenergic Stimulation

CFR remained low (p<0.05) in TAC hearts as isoproterenol concentration increased, as shown in FIG. 18B, maintaining an average of 8.4±0.8 mL/min for all concentrations. Control hearts exhibited substantial vasodilation with increasing isoproterenol concentration. CFR for TAC+OXT hearts was higher than that of TAC hearts at baseline (14±2.9 mL/min) and increased to (15±1.9 mL/min) at the highest isoproterenol concentration. The CFR of Control hearts increased from 13.3±2.4 mL/min at baseline to 25.3±3.7 mL/min at the highest concentration, exhibiting significant CFR reserve. The mean CFR for Control and TAC+OXT hearts was not different, and CFR in both Control and TAC+OXT hearts was significantly higher than TAC hearts (GLM, p<0.05). The RPP response to increased isoproterenol concentration for Control and TAC+OXT hearts was similar for isoproterenol concentrations from baseline until a concentration of 1 nM, as shown in FIG. 18C. Control RPP increased to 52,741±14,328 mmHg*bpm and TAC+OXT RPP increased to 38,017±4,303 mmHg*bpm at the highest isoproterenol concentration. In contrast, TAC hearts only increased to 21,787±3998 mmHg*bpm at the highest concentration.

Changes in LV contractility and relaxation with increasing isoproterenol concentration are shown in FIGS. 19A and 19B. As depicted in FIGS. 19A-19B, hearts from TAC+OXT animals had higher contractility and relaxation; however, contractility and relaxation did not significantly increase with increasing concentrations of isoproterenol. Contractility and relaxation dose-response curves were significantly different between the three groups (p<0.05, GLM and Tukey pairwise analysis). Asterisks indicate significant differences between all groups at specific isoproterenol dose (GLM and Tukey pairwise analysis). As shown in FIG. 19A, Control (n=5) and TAC+OXT (n=7) contractility were similar at isoproterenol concentrations of 1 nM and less. Above 1 nM, the contractility of TAC+OXT hearts did not increase with increasing concentration. The contractility of TAC hearts (n=7) remained low for all concentrations. FIG. 19B shows that Control (n=5) and TAC+OXT (n=7) relaxation were similar at isoproterenol concentrations of 1 nM and less. Above 1 nM, the relaxation of TAC+OXT hearts did not increase with increasing concentration while that of control hearts increased dramatically. The relaxation of TAC hearts (n=7) remained low for all concentrations.

In particular, FIGS. 19A and 19B show that contractility and relaxation were similar in Control and TAC+OXT hearts for isoproterenol concentrations between baseline and 1 nM. At the higher concentrations, the contractility of Control hearts increased to 7,164±788 mmHg/s at the highest isoproterenol concentration. Relaxation of control hearts dropped to −5,402±484 mmHg/s at the highest concentration. Although TAC+OXT hearts initially matched controls, correspondence was lost at concentrations above 1 nM. At the highest concentration, TAC+OXT hearts only reached contractility values of 3,936±589 mmHg/s and relaxation values of −3,258±495 mmHg/s. However, throughout the protocol TAC+OXT contractility and relaxation remained elevated over TAC hearts, which only reached a maximum contractility rate of 2,322±364 mmHg/s and minimum relaxation rate of −2,023±361 mmHg/s, as shown in FIGS. 19A and 19B.

### Discussion

The present example evaluates the effect of chronic activation of PVN OXT neurons in an animal model of pressure overload induced hypertrophy that progresses to HF. The present example also demonstrates the first use of DREADDs to modulate autonomic activity with the goal of mitigating the deleterious effects of cardiac pressure overload. It is generally accepted that parasympathetic tone is cardioprotective and therefore, the present example, demonstrates that chronic activation of PVN OXT neurons confers significant cardioprotection during TAC in rats and demonstrates that PVN OXT neuron activation elevates cardiac parasympathetic tone to alleviate the damaging effects of pressure overload induced hypertrophy.

In ex vivo isovolumic contracting heart studies, it was found that compared to diseased (TAC) animals, the hearts from animals treated with chronic PVN OXT neuron activation (TAC+OXT) developed higher pressures and had greater contractility and relaxation kinetics. Myocyte cross-sectional area was lower and the amount of fibrosis observed in LV tissue histology sections was also lower. These results are consistent with a study of HF following myocardial infarction in which administration of an acetylcholinesterase inhibitor, to increase the duration of muscarinic receptor activation by acetylcholine, improved contractility and reduced myocyte hypertrophy and collagen deposition. In our studies, we also observed that PVN OXT neuron activation during TAC additionally improved cardiac chronotropic response to isoproterenol, as shown in FIG. 18.

Oxytocin may buffer cardiovascular responses to stress and promote cardiac healing by increasing cardiac parasympathetic tone and reducing cardiac sympathetic activation. This was shown by recording in vivo BP and HR responses in DREADDs expressing rats. In such studies, it was discovered that acute activation of DREADDs in PVN OXT neurons reduced both HR and BP. The beneficial effects observed with PVN OXT neuron activation are likely the result of cardiac-specific increases in cholinergic activity. This is supported by the observations that plasma OXT was not different between groups and reductions in BP and HR following DREADDs activation with CNO were completely blocked by atropine, as shown in FIGS. 10 and 14A-B.

The discovery that LV function is significantly improved in TAC+OXT animals after 8 weeks of TAC indicates a sustained beneficial shift in cardiac autonomic balance. In addition to the downstream effects of directly increasing parasympathetic activity, PVN OXT neuron activation may have initiated a parasympathetic mediated reduction in sympathetic activity. Interactions between adrenergic and cholinergic pathways are complex, with multiple factors responsible for activation and antagonism. In the sinus node, cholinergic activation dominates the control of heart rate over that of adrenergic activation. In ventricular myocytes, it is generally accepted that M2 muscarinic receptor activation attenuates the production of cyclic AMP to reduce the inotropic effects of β-adrenergic receptor activation. During chronic sympathetic stimulation, M2 activation reduces myocardial stress by lowering cyclic AMP to reduce the cellular hypercontractile state and increase relaxation during diastole, thereby improving myocyte viability and slowing the progression of hypertrophy. Vagal tone and circulating acetylcholine also maintain beneficial dilation of coronary arteries, an effect that has been shown to be independent of left ventricular preload, afterload, and heart rate. Other studies have shown that parasympathetic nerve stimulation releases endogenous vasoactive intestinal peptide to cause vasodilation and increased coronary flow. Each of these cholinergic-induced outcomes could be cardioprotective during chronic pressure overload and mediated by the activation of PVN OXT neurons.

Left ventricular function was dramatically impaired in untreated TAC animals. The high level of fibrosis and elevated collagen III expression observed in untreated hearts increases wall stiffness, adversely affects contractile function, and negatively impacts vasodilation due to increased myocardial stiffness and increased perivascular collagen. Reduced vasodilation within the context of pressure overload, which increases myocardial oxygen consumption, creates conditions of ischemia, causing further myocardial damage and inflammation. Indeed, we found elevated levels of the inflammatory cytokine IL-1β in TAC animals. As myocytes die, they are replaced by collagen to maintain structural integrity in the absence of cells, which reduces working myocardial mass. Overall, the result of this detrimental cascade was observed in our TAC animals as reduced contractile function, low coronary flow, and a high level of fibrosis.

The present example demonstrates that improved autonomic balance attenuated the loss of cardiac function in the TAC+OXT animals. Although LV hypertrophy was not significantly lower in TAC+OXT animals, likely to compensate for TAC-induced pressure overload, myocyte cross-sectional area was less, fibrosis was less, and there was a lower level of IL-1β expression. The ratio of working myocardium to wall thickness was therefore higher in TAC+OXT than in TAC animals, which likely contributed to the impressive maintenance of LV function that we observed. Improved coronary flow likely augmented the increased myocardial oxygen demand of pressure overload, thereby reducing the incidence of ischemia, preventing myocardial necrosis, preventing the loss of working myocardium, and blunting the progression of myocyte hypertrophy. The interesting finding in TAC+OXT hearts of reduced fibrosis measured via Trichrome staining, yet no significant reduction in collagen III expression, is likely due to increases in perivascular collagen. Increased perivascular collagen would not have been detected in the myocardial fibrosis assessments that were conducted using Trichrome-stained myocardial slices. However, increased perivascular collagen would elevate the level of collagen III measured in the western blot assays.

The present example demonstrates that PVN OXT neuron activation in TAC animals partially blunts sinus node desensitization to β-adrenergic stimulation. The result that TAC+OXT animals had improved HR sensitivity to β-adrenergic stimulation, but no improvement in contractile sensitivity, provides new insight into how the impact of heterogeneous cardiac nerve density may affect the outcomes of parasympathetic nerve activation. The dense innervation of cholinergic nerve fibers in the atria and sinus node, as well as greater expression of M2 receptors in the atria compared to the ventricles, at least partially explains the observed differences in HR and LV contractile response of TAC+OXT animals to adrenergic stimulation, as shown in FIGS. 18 and 19.

Although the contractility and relaxation kinetics of hearts from TAC+OXT animals, for all isoproterenol concentrations, was greater than that of hearts from TAC animals, it is intriguing that there was almost no increase in these values as isoproterenol concentration increased. Indeed, cholinergic nerve fibers and muscarinic receptors are found in the ventricles of many species, including rodents. One explanation for the observed result could be a lower ratio of ventricular parasympathetic to sympathetic innervation and a lower expression of M2 receptors in the ventricles compared to β-receptors. The ratio of cholinergic to adrenergic innervation is close to 2:1 in the atria and 1:2 in the ventricles. Within the context of this intrinsic anatomic imbalance between cholinergic and adrenergic activation of the LV, and the elevation of sympathetic tone that occurs during chronic pressure overload, our results indicate that PVN OXT neuron activation is not enough to halt ventricular adrenergic desensitization.

The present example demonstrates that chronic activation of PVN OXT neurons, beginning 4 weeks after TAC, significantly improved LV function, including inotropy and lusitropy, and reduced cellular hypertrophy, fibrosis, and inflammation at 8 weeks after TAC. PVN OXT neuron activation also improved heart rate sensitivity to β-adrenergic stimulation but did not improve contractile sensitivity to β-adrenergic stimulation. The present example demonstrates that the selective activation of hypothalamic PVN OXT neurons may be an effective approach to counteract the loss of cardiac function and mitigate myocardial damage during pressure overload hypertrophy.

### Example 2

### Intranasal Administration of Oxytocin Improves Clinical Outcomes in Subjects Diagnosed with Heart Failure

The effect of intranasal administration of oxytocin in the treatment of human subjects having heart failure will be studied using a randomized double blinded cross-over study. Twenty subjects that have recently been diagnosed with moderate heart failure (HF, NYHA II-III patients and those with LVEF ˜30%) will be enrolled in this FDA approved randomized double-blinded controlled trial. Subjects will be randomized by the pharmacy to be administered either 40 IU of oxytocin twice daily or placebo (sterile water spray, twice daily) for the initial six month period. In the second six month period, the subjects will crossover between treatment and the placebo group. For example, if a subject is randomized to receive placebo for the first six months, upon completion of this six month period, the subject will now self-administer oxytocin for the next six months. If a subject is randomized to receive oxytocin for the first six months of the trial, then he or she will receive placebo for the final six months.

Subjects will be at least 18 years old and have functional NYHA class II or III heart failure (patients with heart disease resulting in slight limitation of physical activity; symptoms of HF develop with ordinary activity but there are no symptoms at rest). Subjects will also have no hospital admissions in the last month and be currently receiving medical management according to the current ACC/AHA guidelines. All subjects will be optimized by cardiology on their best medical therapy based on American College of Cardiology and American Heart Association guidelines. Patients who have been hospitalized within the last month, history of myocardial infarction the last three months, history of chronic kidney disease, history of cirrhosis, asthma, chronic obstructive pulmonary disease, current smoker, or unable to participate in 6-minute walk test due to mobility issues will be excluded from the study. If subjects are female and of childbearing potential, then their birth control method for the duration of the study will be recorded, and a urine pregnancy test will be performed. If positive, the patient will be excluded from the study. All interviews and assessment will be carried out in English, and therefore if the patient is unable to participate, they will be excluded.

If the subject consents to the study, he or she will be randomized by the pharmacy using a computer program to be administered either Oxytocin (40 IU, twice daily) or placebo (sterile water spray, twice daily) for the initial six month period. In the second six month period, the subjects will crossover to the intervention that they did not receive during the first six month period of study. All of the subjects will be followed for an additional three months after they have received both, the placebo and the Oxytocin. Detailed clinical information will be recorded with a specific focus on the six major outcomes, which include ejection fraction (transthoracic echocardiogram (TTE)), pro-BNP, number of hospital admissions, symptoms (chest pain, dyspnea, palpitations, orthopnea, paroxysmal nocturnal dyspnea), electrocardiogram (EKG), and a 6-minute walk test. Subjects will be evaluated at baseline, three months, six months, nine months and twelve months.

Subjects that self-administer intranasal oxytocin treatment are expected to have improved standard clinical outcomes and indices of heart failure. In particular, subjects receiving intranasal oxytocin are expected to exhibit an improvement in one or more of the following clinical outcomes: cardiac function as determined by transthoracic echocardiogram (TTE), exercise or stress tolerance, dyspnea, ejection fraction, fractional shortening velocity, relaxation velocity, reduced incidence of arrhythmias and improved systolic (contractile) and/or diastolic (relaxation) function.

### Example 3

### Intranasal Administration of Oxytocin Improves Clinical Outcomes in Subjects Diagnosed with Heart Failure with Reduced Ejection Fraction (HFrEF)

The effect of intranasal administration of oxytocin in the treatment of human subjects having heart failure with reduced ejection fraction with will be studied using the randomized double blinded cross-over study described in Example 1 above. All subjects will have an ejection fraction less than 45% according to transthoracic echocardiogram (TTE) based on American Society of Echocardiography guidelines. Subjects having heart failure with reduced ejection fraction that self-administer intranasal oxytocin treatment are expected to have improved standard clinical outcomes and indices of heart failure. In particular, subjects receiving intranasal oxytocin are expected to exhibit an improvement in one or more of the following clinical outcomes: cardiac function as determined by transthoracic echocardiogram (TTE), exercise or stress tolerance, dyspnea, ejection fraction, fractional shortening velocity, relaxation velocity, reduced incidence of arrhythmias and improved systolic (contractile) and/or diastolic (relaxation) function.

### Example 4

### Intranasal Administration of Oxytocin Improves Clinical Outcomes in Subjects Diagnosed with Heart Failure without Ischemic Heart Disease

The effect of intranasal administration of oxytocin in the treatment of human subjects having heart failure but that do not have ischemic heart disease will be studied using the randomized double blinded cross-over study described in Example 1 above. Subjects having heart failure without ischemic heart disease that self-administer intranasal oxytocin treatment are expected to have improved standard clinical outcomes and indices of heart failure. In particular, subjects receiving intranasal oxytocin are expected to exhibit an improvement in one or more of the following clinical outcomes: cardiac function as determined by transthoracic echocardiogram (TTE), exercise or stress tolerance, dyspnea, ejection fraction, fractional shortening velocity, relaxation velocity, reduced incidence of arrhythmias and improved systolic (contractile) and/or diastolic (relaxation) function.

### Example 5

### Intranasal Administration of Oxytocin Improves Clinical Outcomes in Subjects Diagnosed with Heart Failure with Preserved Ejection Fraction

The effect of intranasal administration of oxytocin in the treatment of human subjects having heart failure with preserved ejection fraction will be studied using the randomized double blinded cross-over study described in Example 1 above. All subjects will have a left ventricular ejection fraction that is greater than 50%. Subjects having heart failure with preserved ejection fraction that self-administer intranasal oxytocin treatment are expected to have improved standard clinical outcomes and indices of heart failure. In particular, subjects receiving intranasal oxytocin are expected to exhibit an improvement in one or more of the following clinical outcomes: cardiac function as determined by transthoracic echocardiogram (TTE), exercise or stress tolerance, dyspnea, ejection fraction, fractional shortening velocity, relaxation velocity, reduced incidence of arrhythmias and improved systolic (contractile) and/or diastolic (relaxation) function.

### Example 6

### Intranasal Administration of Oxytocin Improves Clinical Outcomes in Subjects Diagnosed with Left Ventricular Hypertrophy

The effect of intranasal administration of oxytocin in the treatment of human subjects having left ventricular hypertrophy will be studied using the randomized double blinded cross-over study described in Example 1 above. All subjects will have walls of the left ventricle of their heart greater than 1.5 centimeters as measured by echocardiogram. Subjects having left ventricular hypertrophy that self-administer intranasal oxytocin treatment are expected to have improved standard clinical outcomes and indices of heart failure. In particular, subjects receiving intranasal oxytocin are expected to exhibit an improvement in one or more of the following clinical outcomes: cardiac function as determined by transthoracic echocardiogram (TTE), exercise or stress tolerance, dyspnea, ejection fraction, fractional shortening velocity, relaxation velocity, reduced incidence of arrhythmias and improved systolic (contractile) and/or diastolic (relaxation) function.

Statements of the Disclosure Include:

Statement 1: A method for treating or delaying heart failure in a subject in need thereof, the method comprising administering intranasally to the subject a therapeutically effective amount of oxytocin.

Statement 2: A method for slowing or arresting heart failure in a subject in need thereof, the method comprising administering intranasally to the subject a therapeutically effective amount of oxytocin.

Statement 3: A method of treating a subject having heart failure, the method comprising administering intranasally to the subject a therapeutically effective amount of oxytocin.

Statement 4: A method according to any one of the preceding Statements 1-3, wherein the therapeutically effective amount is from about 20 IU to about 100 IU.

Statement 5: A method according to any one of the preceding Statements 1-4, wherein the therapeutically effective amount is administered twice per day.

Statement 6: A method according to any one of the preceding Statements 1-3, wherein the pharmaceutically effective amount is from about 20 to about 40 IU b.i.d.

Statement 7: A method according to any one of the preceding Statements 1-6, wherein the pharmaceutically effective amount is administered at least once per day for at least 5 days.

Statement 8: A method according to any one of the preceding Statements 1-6, wherein the pharmaceutically effective amount is administered at least twice per day for at least 5 days.

Statement 9: A method according to any one of the preceding Statements 1-6, wherein the pharmaceutically effective amount is administered at least once per day for consecutive days.

Statement 10: A method according to any one of the preceding Statements 1-6, wherein the pharmaceutically effective amount is administered at least twice per day for consecutive days.

Statement 11: A method according to any one of the preceding Statements 1-10, wherein the subject has hypertrophy of the heart.

Statement 12: A method according to any one of the preceding Statements 1-11, wherein the subject has left ventricular hypertrophy (LVH).

Statement 13: A method according to any one of the preceding Statements 1-12, wherein the subject has cardiac ischemia.

Statement 14: A method according to any one of the preceding Statements 1-12, wherein the subject does not have ischemic heart disease.

Statement 15: A method according to any one of the preceding Statements 1-14, wherein the subject is diagnosed with a left ventricular ejection fraction of less than or equal to 40%.

Statement 16: A method according to any one of the preceding Statements 1-15, wherein heart failure comprises NYHA Class II or NYHA Class III heart failure.

Statement 17: A method according to any one of the preceding Statements 1-16, wherein heart failure comprises heart failure with reduced ejection fraction.

Statement 18: A method according to any one of the preceding Statements 1-16, wherein heart failure comprises heart failure with preserved ejection fraction.

Statement 19: A method according to any one of the preceding Statements 1-16, wherein the heart failure is left ventricular hypertrophy-induced heart failure.

Statement 20: A method according to any one of the preceding Statements 1-19, further comprising administering to the subject a therapeutically effective amount of at least one of the group consisting of nitric oxide, atrial natriuretic peptide (ANP), and beta-blockers.

Statement 21: A method according to any one of the preceding Statements 1-20, wherein the subject is a mammal.

Statement 22: A method according to any one of the preceding Statements 1-20, wherein the subject is a human subject.

Statement 23: A method of treating a subject diagnosed with heart failure, the method comprising activating hypothalamic oxytocin neurons in the brain of the subject.

Statement 24: A method according to Statement 23, wherein activating hypothalamic oxytocin neurons in the brain of the subject comprises chronic activation of PVN OXT neurons.

Statement 25: A method according to Statement 23 or Statement 24, wherein activating hypothalamic oxytocin neurons in the brain of the subject comprises administering an effective amount of oxytocin to the subject.

Statement 26: A method according to Statement 25, wherein administering an effective amount of oxytocin comprises intranasal administration of oxytocin to the subject.

Statement 27: A method according to Statement 25 or Statement 26, wherein the therapeutically effective amount is from about 20 IU to about 100 IU.

Statement 28: A method according to any one of the preceding Statements 25-27, wherein the therapeutically effective amount is administered twice per day.

Statement 29: A method according to any one of the preceding Statements 25-28, wherein the pharmaceutically effective amount is from about 20 to about 40 IU b.i.d.

Statement 30: A method according to any one of the preceding Statements 25-29, wherein the pharmaceutically effective amount is administered at least once per day for at least 5 days.

Statement 31: A method according to any one of the preceding Statements 25-29, wherein the pharmaceutically effective amount is administered at least twice per day for at least 5 days.

Statement 32: A method according to any one of the preceding Statements 25-27, wherein the pharmaceutically effective amount is administered at least once per day for consecutive days.

Statement 33: A method according to any one of the preceding Statements 25-27, wherein the pharmaceutically effective amount is administered at least twice per day for consecutive days.

Statement 34: A method according to any one of the preceding Statements 23-33, wherein the subject has hypertrophy of the heart.

Statement 35: A method according to any one of the preceding Statements 23-34, wherein the subject has left ventricular hypertrophy (LVH).

Statement 36: A method according to any one of the preceding Statements 23-35, wherein the subject has cardiac ischemia.

Statement 37: A method according to any one of the preceding Statements 23-35, wherein the subject does not have ischemic heart disease.

Statement 38: A method according to any one of the preceding Statements 23-37, wherein the subject is diagnosed with a left ventricular ejection fraction of less than or equal to 40%.

Statement 39: A method according to any one of the preceding Statements 23-38, wherein heart failure comprises NYHA Class II or NYHA Class III heart failure.

Statement 40: A method according to any one of the preceding Statements 23-38, wherein heart failure comprises heart failure with reduced ejection fraction.

Statement 41: A method according to any one of the preceding Statements 23-38, wherein heart failure comprises heart failure with preserved ejection fraction.

Statement 42: A method according to any one of the preceding Statements 23-38, wherein the heart failure is left ventricular hypertrophy-induced heart failure.

Statement 43: A method according to any one of the preceding Statements 23-42, further comprising administering to the subject a therapeutically effective amount of at least one of the group consisting of nitric oxide, atrial natriuretic peptide (ANP), and beta-blockers.

Statement 44: A method according to any one of the preceding Statements 23-43, wherein the subject is a mammal.

Statement 45: A method according to any one of the preceding Statements 23-43, wherein the subject is a human subject.

Statement 46: A method of treating a subject having left ventricular hypertophy, the method comprising administering intranasally to the subject a therapeutically effective amount of oxytocin.

Statement 47: A method of treating a subject having heart failure without ischemic heart disease, the method comprising administering intranasally to the subject a therapeutically effective amount of oxytocin.

Statement 48: A method of improving cardiac function in a subject in need thereof, the method comprising administering intranasally to the subject a therapeutically effective amount of oxytocin.

Statement 49: A method of improving cardiac contractile performance in a subject in need thereof, the method comprising administering intranasally to the subject a therapeutically effective amount of oxytocin.

Statement 50: A method according to any of the preceding Statements 36-49, wherein the therapeutically effective amount is from about 20 IU to about 100 IU.

Statement 51: A method according to any one of the preceding Statements 36-50, wherein the therapeutically effective amount is administered twice per day.

Statement 52: A method according to any one of the preceding Statements 36-51, wherein the pharmaceutically effective amount is from about 20 to about 40 IU b.i.d.

Statement 53: A method according to any one of the preceding Statements 36-52, wherein the pharmaceutically effective amount is administered at least once per day for at least 5 days.

Statement 54: A method according to any one of the preceding Statements 36-52, wherein the pharmaceutically effective amount is administered at least twice per day for at least 5 days.

Statement 55: A method according to any one of the preceding Statements 36-52, wherein the pharmaceutically effective amount is administered at least once per day for consecutive days.

Statement 56: A method according to any one of the preceding Statements 36-52, wherein the pharmaceutically effective amount is administered at least twice per day for consecutive days.

Statement 57: A method according to any one of the preceding Statements 36-56, wherein the subject has hypertrophy of the heart.

Statement 58: A method according to any one of the preceding Statements 36-57, wherein the subject has left ventricular hypertrophy (LVH).

Statement 59: A method according to any one of the preceding Statements 36-58, wherein the subject has cardiac ischemia.

Statement 60: A method according to any one of the preceding Statements 36-58, wherein the subject does not have ischemic heart disease.

Statement 61: A method according to any one of the preceding Statements 36-60, wherein the subject is diagnosed with a left ventricular ejection fraction of less than or equal to 40%.

Statement 62: A method according to any one of the preceding Statements 36-61, wherein heart failure comprises NYHA Class II or NYHA Class III heart failure.

Statement 63: A method according to any one of the preceding Statements 36-61, wherein heart failure comprises heart failure with reduced ejection fraction.

Statement 64: A method according to any one of the preceding Statements 36-61, wherein heart failure comprises heart failure with preserved ejection fraction.

Statement 65: A method according to any one of the preceding Statements 36-61, wherein the heart failure is left ventricular hypertrophy-induced heart failure.

Statement 66: A method according to any one of the preceding Statements 36-65, further comprising administering to the subject a therapeutically effective amount of at least one of the group consisting of nitric oxide, atrial natriuretic peptide (ANP), and beta-blockers.

Statement 67: A method according to any one of the preceding Statements 36-66, wherein the subject is a mammal.

Statement 68: A method according to any one of the preceding Statements 36-66, wherein the subject is a human subject.

Statement 69: An intranasal formulation for the treatment of a subject diagnosed with heart failure, the intranasal formulation comprising a therapeutically effective amount of oxytocin, wherein the formulation is capable of being delivered intranasally to the subject.

Statement 70: An intranasal formulation according to Statement 69, further comprising a pharmaceutically acceptable carrier.

Statement 71: An intranasal formulation according to Statement 70, wherein the pharmaceutically acceptable carrier is selected from the group consisting of water, ethanol, propylene glycol, polyethylene glycol, vegetable oils, organic esters, glycerin, phenol, dimethyl sulfoxide, N-tridecyl-β-D-maltoside, and any combination thereof.

Statement 72: An intranasal formulation according to any one of the preceding Statements 69-71, wherein the formulation is manufactured to supply oxytocin in an amount from about 10 IU to about 100 IU per each administration.

Statement 73: An intranasal formulation according to any one of the preceding Statements 69-71, wherein the formulation is manufactured to supply oxytocin in an amount from about 20 IU to about 40 IU per each administration.

Statement 74: An intranasal formulation according to any one of the preceding Statements 69-71, wherein the formulation is manufactured to supply oxytocin in an amount from about 20 IU to about 40 IU b.i.d.

Statement 75: An intranasal formulation according to any one of the preceding Statements 69-71, wherein the formulation is manufactured to supply oxytocin in an amount of about 40 IU per each administration.

Statement 76: An intranasal formulation according to any one of the preceding Statements 69-75, wherein the formulation is manufactured to be administered twice per day.

Statement 77: An intranasal formulation according to any one of the preceding Statements 69-76, wherein the subject has hypertrophy of the heart.

Statement 78: An intranasal formulation according to any one of the preceding Statements 69-77, wherein the subject has left ventricular hypertrophy (LVH).

Statement 79: An intranasal formulation according to any one of the preceding Statements 69-78, wherein the subject has cardiac ischemia.

Statement 80: An intranasal formulation according to any one of the preceding Statements 69-78, wherein the subject does not have ischemic heart disease.

Statement 81: An intranasal formulation according to any one of the preceding Statements 69-80, wherein the subject is diagnosed with a left ventricular ejection fraction of less than or equal to 40%.

Statement 82: An intranasal formulation according to any one of the preceding Statements 69-81, wherein heart failure comprises NYHA Class II or NYHA Class III heart failure.

Statement 83: An intranasal formulation according to any one of the preceding Statements 69-81, wherein heart failure comprises heart failure with reduced ejection fraction.

Statement 84: An intranasal formulation according to any one of the preceding Statements 69-81, wherein heart failure comprises heart failure with preserved ejection fraction.

Statement 85: An intranasal formulation according to any one of the preceding Statements 69-81, wherein the heart failure is left ventricular hypertrophy-induced heart failure.

Statement 86: An intranasal formulation according to any one of the preceding Statements 69-85, further comprising a therapeutically effective amount of at least one of the group consisting of nitric oxide, atrial natriuretic peptide (ANP), and beta-blockers.

Statement 87: An intranasal formulation according to any one of the preceding Statements 69-86, wherein the subject is a mammal.

Statement 88: An intranasal formulation according to any one of the preceding Statements 69-86, wherein the subject is a human subject.

Statement 89: A method according to any one of the preceding Statements 23-24 or 34-45, wherein activating hypothalamic oxytocin neurons in the brain of the subject comprises causing viral mediated expression of exogenous receptors, said exogenous receptors activatable by an inert biological agent; the method further comprising administering to the subject an inert biological agent capable of activating the exogenous receptors.

We claim:

1. A method of treating a subject having heart failure, the method comprising administering intranasally to the subject a therapeutically effective amount of oxytocin.

2. The method according to claim 1, wherein the therapeutically effective amount is from about 20 IU to about 100 IU.

3. The method according to claim 2, wherein the therapeutically effective amount is administered twice per day.

4. The method according to claim 1, wherein the pharmaceutically effective amount is from about 20 to about 40 IU b.i.d.

5. The method according to claim 1, wherein the subject has left ventricular hypertrophy (LVH).

6. The method according to claim 1, wherein the subject does not have ischemic heart disease.

7. The method according to claim 1, wherein heart failure comprises one selected from the group consisting of NYHA Class II or NYHA Class III heart failure, heart failure with reduced ejection fraction, heart failure with preserved ejection fraction, and left ventricular hypertrophy-induced heart failure.

8. The method according to claim 1, further comprising administering to the subject a therapeutically effective amount of at least one of the group consisting of nitric oxide, atrial natriuretic peptide (ANP), and beta-blockers.

9. The method according to claim 1, wherein the subject is a mammal or a human subject.

10. A method of treating a subject diagnosed with heart failure, the method comprising activating hypothalamic oxytocin neurons in the brain of the subject.

11. The method according to claim 1, wherein activating hypothalamic oxytocin neurons in the brain of the subject comprises chronic activation of PVN OXT neurons.

12. The method according to claim 1, wherein activating hypothalamic oxytocin neurons in the brain of the subject comprises intranasally administering an effective amount of oxytocin to the subject.

13. The method according to claim 12, wherein the pharmaceutically effective amount is from about 20 to about 40 IU b.i.d.

14. The method according to claim 10, wherein the subject has left ventricular hypertrophy (LVH).

15. The method according to claim 10, wherein heart failure comprises one selected from the group consisting of NYHA Class II or NYHA Class III heart failure, heart failure with reduced ejection fraction, heart failure with preserved ejection fraction, and left ventricular hypertrophy-induced heart failure.

16. An intranasal formulation for the treatment of a subject diagnosed with heart failure, the intranasal formulation comprising a therapeutically effective amount of oxytocin, wherein the formulation is capable of being delivered intranasally to the subject.

17. The intranasal formulation according to claim 16, further comprising a pharmaceutically acceptable carrier.

18. The intranasal formulation according to claim 17, wherein the pharmaceutically acceptable carrier is selected from the group consisting of water, ethanol, propylene glycol, polyethylene glycol, vegetable oils, organic esters, glycerin, phenol, dimethyl sulfoxide, N-tridecyl-β-D-maltoside, and any combination thereof.

19. The intranasal formulation according to claim 1, wherein the formulation is manufactured to supply oxytocin in an amount from about 10 IU to about 100 IU per each administration.

20. The intranasal formulation according to claim 1, wherein the formulation is manufactured to supply oxytocin in an amount from about 20 IU to about 40 IU per each administration.
